(19) United States
(12) Patent Application Publication
(10) Pub. No.: US 2025/0143579 A1
(43) Pub. Date: May 8, 2025

(54) Detection Device

(71) Applicant: Advanced ACEBIOTEK Co., Ltd., Hsinchu County (TW)

(72) Inventors: Jyh-Chern Chen, New Taipei City (TW); Huan-Pin Tai, Hsinchu City (TW); Yung-Chou Hsu, New Taipei City (TW); Hui-Shun Kuan, Taipei City (TW); Shen-Fu Hsu, Taitung County (TW)

(73) Assignee: Advanced ACEBIOTEK Co., Ltd., Hsinchu County (TW)

(21) Appl. No.: 18/528,759

(22) Filed: Dec. 4, 2023

(30) Foreign Application Priority Data
Nov. 3, 2023 (TW) 112142447

(51) Int. Cl. A61B 5/00

(52) U.S. Cl. CPC A61B 5/0084; A61B 5/6852; A61B 2562/223

(57) ABSTRACT

A detection device for an optical detection system comprises an optical fiber catheter, having a feed opening and a dome end; and an optical fiber detector, having one end coupled to the optical detection system through a connection terminal, and another end capable of entering an interior of the optical fiber catheter through the feed opening of the optical fiber catheter and reaching the dome end, configured to guide detection light beams generated by the optical detection system to the dome end, and guide reaction light beams corresponding to the detection light beams from the dome end to the optical detection system.

### BACKGROUND OF THE INVENTION

### 1. Field of the Invention

The present invention relates to a detection device, and more particularly, to a detection device capable of reducing the discomfort of the testee and the difficulty of detection.

### 2. Description of the Prior Art

A mucous membrane tissue, also known as mucosal tissue, forms a part of surface tissues of various organs in a human body exposed to the external environment. The mucosal tissue is a membranous structure composed of epithelial tissues and connective tissues. The mucosal tissue is a crucial part of the human body to interact with various antigens found in nature. Not only does the mucosal tissue contribute to the functioning of organs, but it also serves as the first line of defense for the human immune system. There are several conventional test methods to determine the health of mucosal tissues, including checking for inflammation, cancer, abnormal cell proliferation, or tumors. These methods may involve computed tomography scans, nuclear magnetic resonance (NMR), ultrasonic tomography, optical coherence tomography, X-rays, endoscopy, and pathology. Endoscopy is a commonly used early detection method for serious diseases, but it still has limitations such as poor image resolution, the need for anesthesia or local gas inflation of the organ cavity, and the requirement of a contrast agent, which may not be suitable for patients who experiences side effects from the contrast agent. Further, optical biopsy methods have gained attention in the field of biomedicine due to their advantages, such as not requiring the collection of human tissue specimens and providing real-time imaging to assist in medical diagnosis. However, the results of conventional optical biopsy diagnosis methods are still based on the image information of the mucosal surface. Consequently, the clinical professionals may be prone to make erroneous judgments or have differing opinions when diagnosing based on the image information.

In such a situation, the applicant has proposed an optical detection system in US 2023/0059771, which can calculate health parameters through the target spectral signal corresponding to the target tissue area and the reference spectral signal corresponding to the reference tissue area, providing real-time detection. Therefore, the optical detection system of US 2023/0059771 can be widely used in the detection of mucosal tissues. For example, the health status of endometrial mucosal tissue is closely related to many gynecological diseases. Through the optical detection system of US 2023/0059771, the light beams can be used to enter the uterus through the vagina using optical fiber, and the reaction spectrum of the endometrium illuminated by the light beams can be measured to assess the health status of the endometrial mucosal tissue. However, due to the complex structure of the passage from the vagina through the cervical canal to the inside of the uterus, improper use of instruments or larger diameter will easily scratch the vagina, cervix and uterine cavity, causing bleeding. Especially for those with cervical canal deformities, it further increases the difficulty of entering the uterine cavity through the deformed cervical canal. Therefore, how to design an optical fiber detector of endometrium that is easy to enter the uterine cavity through the cervical canal has become one of the goals of the industry.

### SUMMARY OF THE INVENTION

Therefore, the present invention is to provide a detection device for an optical detection system to reduce the discomfort of the testee and the difficulty of detection.

An embodiment of the present invention discloses a detection device for an optical detection system, which comprises an optical fiber catheter, having a feed opening and a dome end; and an optical fiber detector, having one end coupled to the optical detection system through a connection terminal, and another end capable of entering an interior of the optical fiber catheter through the feed opening of the optical fiber catheter and reaching the dome end, configured to guide detection light beams generated by the optical detection system to the dome end, and guide reaction light beams corresponding to the detection light beams from the dome end to the optical detection system.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a detection device according to an embodiment of the present invention.

FIG. 2 to FIG. 4 are schematic diagrams of the combination of an optical fiber detector and an optical fiber catheter shown in FIG. 1.

FIG. 5 is a schematic diagram of the application of the detection device of FIG. 1 to the optical detection system of US 2023/0059771 to realize the fiber module.

### DETAILED DESCRIPTION

Certain terms are used throughout the description and following claims to refer to particular components. As one skilled in the art will appreciate, hardware manufacturers may refer to a component by different names. This document does not intend to distinguish between components that differ in name but not function. In the following description and in the claims, the terms “include” and “comprise” are utilized in an open-ended fashion, and thus should be interpreted to mean “include, but not limited to . . . ”. Also, the term “couple” is intended to mean either an indirect or direct electrical connection. Accordingly, if one device is coupled to another device, that connection may be through a direct electrical connection, or through an indirect electrical connection via other devices and connections.

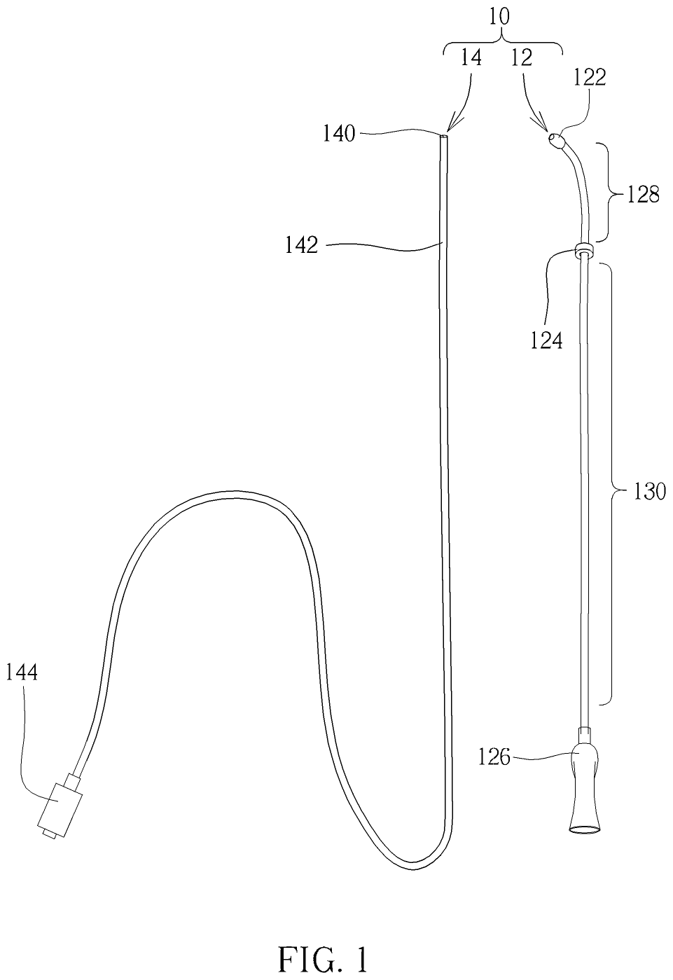

Please refer to FIG. 1, which is a schematic diagram of a detection device 10 according to an embodiment of the present invention. The detection device 10 may be used in an optical detection system, such as the optical detection system disclosed in US 2023/0059771, to guide and transmit the light generated by the light source device of the optical detection system to irradiate the test area of the human body, especially the human mucosal tissue, such as endometrial mucosal tissue. Due to the complex structure of the passage from the vagina through the cervical canal to the inside of the uterus, improper use of instruments or larger diameter will easily scratch the vagina, cervix and uterine cavity, causing bleeding, and especially for those with cervical canal deformities, it further increases the difficulty of entering the uterine cavity through the deformed cervical canal. In this regard, the detection device 10 of the present invention may be applied to the optical detection system to conduct light and irradiate the target tissue area of the human body, and receive the reaction beams from the target tissue area. At the same time, the detection device 10 of the present invention has special structures, which can easily enter the uterine cavity through the cervical canal, reduce the discomfort of the testee, and reduce the difficulty of detection.

Specifically, the detection device 10 includes an optical fiber catheter 12 and an optical fiber detector 14. The optical fiber detector 14 may be combined with the optical fiber catheter 12 through the inside of the optical fiber catheter 12 to perform detection with the optical detection system. The optical fiber catheter 12 is a hollow conduit, which includes a feed opening 120 and a dome end 122, and is equipped with a positioning ring 124 and a handle 126. The positioning ring 124 is set on the optical fiber catheter 12, forming a detection tube section 128 with the dome end 122, and forming an extension tube section 130 with the feed opening 120. The positioning ring 124 may move along the optical fiber catheter 12 to adjust the relative lengths of the detection tube section 128 and the extension tube section 130. The handle 126 is set on a position of the optical fiber catheter 12 near the feed opening 120, roughly in the shape of a gourd with larger ends and a smaller middle, and includes a rough surface for easy handheld operation by the inspector.

On the other hand, the optical fiber detector 14 includes an optical fiber bundle 140 and an optical fiber bundle sheath 142, and is coupled to the optical detection system through a connection terminal 144. In one embodiment, the optical fiber bundle 140 includes multiple optical fibers, such as 3, and the connection terminal 144 includes optical fiber connectors respectively corresponding to the optical fibers. The optical fiber bundle sheath 142 is used to cover and protect the optical fiber bundle 140, which can bundle the optical fibers in the optical fiber bundle 140 closely together for easy passage through the feed opening 120. In addition, depending on the design of the optical detection system, the optical fiber connectors corresponding to each optical fiber in the connection terminal 144 can be separable or integrated; if they are separable, the optical fiber bundle sheath 142 needs to be able to fan out the optical fibers connected to the optical fiber connectors for easy connection to the optical detection system.

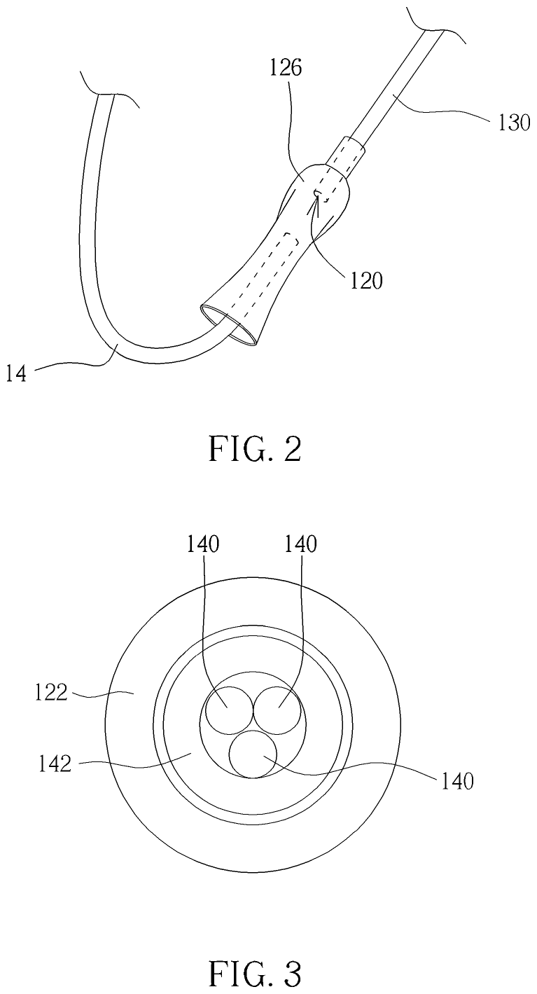

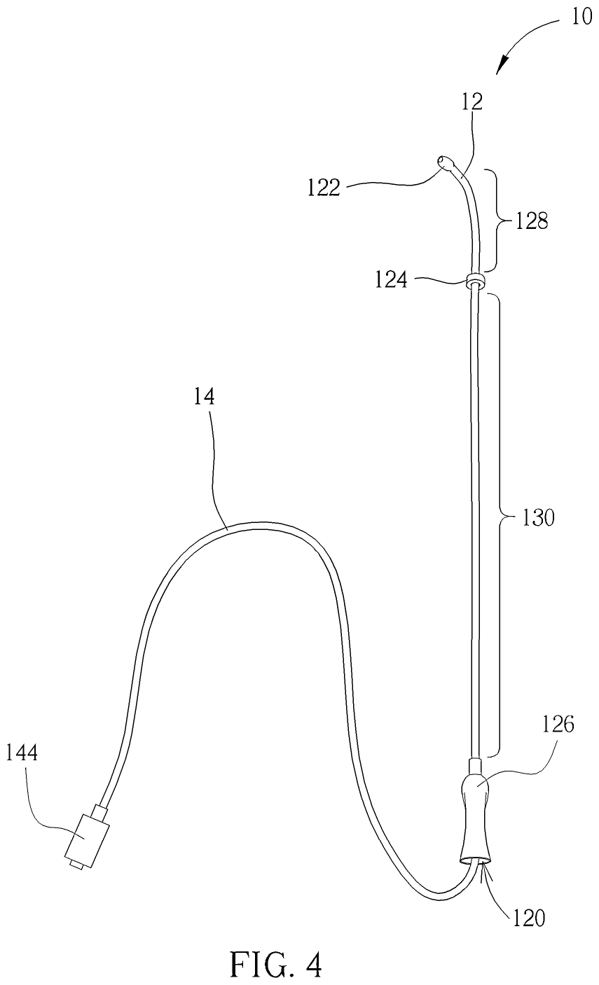

Please continue to refer to FIG. 2 to FIG. 4, which are schematic diagrams of the combination of the optical fiber detector 14 and the optical fiber catheter 12. As shown in FIG. 2, when using the detection device 10 for detection, the inspector should insert the optical fiber detector 14 (by an end not connected to the connection terminal 144) into the interior of the optical fiber catheter 12 through the handle 126 and the feed opening 120, and continue to advance to reach the dome end 122. FIG. 3 is a cross-sectional view of the dome end 122 after the optical fiber detector 14 and the optical fiber catheter 12 are combined. As can be seen from FIG. 3, the optical fiber bundle sheath 142 bundles the optical fibers in the optical fiber bundle 140 closely together to pass through the feed opening 120, and finally reaches the dome end 122. FIG. 4 is the result of the combination of the optical fiber detector 14 and the optical fiber catheter 12. In this way, the inspector can hold the handle 126 to inspect the testee.

In detail, when using the detection device 10 in conjunction with the optical detection system for detection, the inspector should couple the connection terminal 144 to the optical detection system. The inspector may adjust the position of the positioning ring 124, for example, first determine the target tissue area of the testee (such as the position of the cervix, the length of the cervical canal, the length of the vagina, etc.) through a supersonic diagnostic set, and adjust the length of the detection tube section 128 through the positioning ring 124. Then, as shown in FIG. 2 to FIG. 4, the inspector may insert the optical fiber detector 14 into the interior of the optical fiber catheter 12 through the handle 126 and the feed opening 120, and continue to advance to reach the dome end 122. After the optical fiber detector 14 is combined with the optical fiber catheter 12, the inspector may hold the handle 126 to inspect the testee. It should be noted that, as shown in FIG. 1 or FIG. 4, the detection tube section 128 includes a bend, making it easy to enter the uterine cavity through the cervical canal, while the extension tube section 130 is straight, making it easy for the inspector to operate. In different embodiments, the bending angle of the detection tube section 128 may be selected from one of 0° to 15°, 15° to 30°, 30° to 45°, 45° to 60°, 60° to 75°, and 75° to 90°, but not limited to this, and the material of the optical fiber catheter 12 may be appropriately selected to make the detection tube section 128 flexible and soft, and make the extension tube section 130 straight (or rigid). In addition, the shape of the handle 126 is not limited to a gourd shape, and its opening may be aligned with the feed opening 120, or protrude from the feed opening 120, or the handle 126 may also move on the optical fiber catheter 12, etc., all of which are applicable to the present invention, as long as it does not obstruct the optical fiber detector 14 from passing through the feed opening 120.

In one embodiment, the diameter of the optical fiber catheter 12 is smaller than or equal to 3.0 millimeters, the total length thereof is greater than 5 centimeters, and at least one scale indicator may be formed on the optical fiber catheter 12, so that the inspector may judge the depth, but not limited to this. In one embodiment, the outer diameter of the positioning ring 124 is smaller than or equal to 6 millimeters. In one embodiment, the dome end 122 is a hollow smooth spherical shape, and the ball diameter thereof is smaller than or equal to 3.0 millimeters. The dome end 122 may have a transparent film in the top side and may prevent the optical fiber detector 14 from protruding from the dome end 122. In one embodiment, the dome end 122, the detection tube section 128, the positioning ring 124, and the extension tube section 130 of the optical fiber catheter 12 all have a smooth surface. In one embodiment, the optical fiber catheter 12 is made of one or more of polytetrafluoroethylene, silicon, polyethylene, polypropylene, polymethylpentene, or polyvinyl chloride. On the other hand, in the optical fiber detector 14, the optical fibers contained in the optical fiber bundle 140 are preferably step-index multimode optical fibers, which include an optical fiber core, an optical fiber cladding, and a protective coating, but not limited to this. In one embodiment, the optical detection system may transmit the generated detection light beams to at least one optical fiber of the optical fiber bundle 140 through the connector terminal 144, and then guide the beams to the dome end 122 to emit to the test area; the other optical fibers may guide the reaction light beams corresponding to the detection light beams from the dome end 122 to the corresponding connectors for output to the optical detection system. In addition, the detection device 10 is preferably appropriately packaged after sterilization, and all components of the detection device 10 may be disposable components for one-time use, or components for repeated use after sterilization, or some components are for one-time use and other components are for repeated use, etc., which are all within the scope of the present invention.

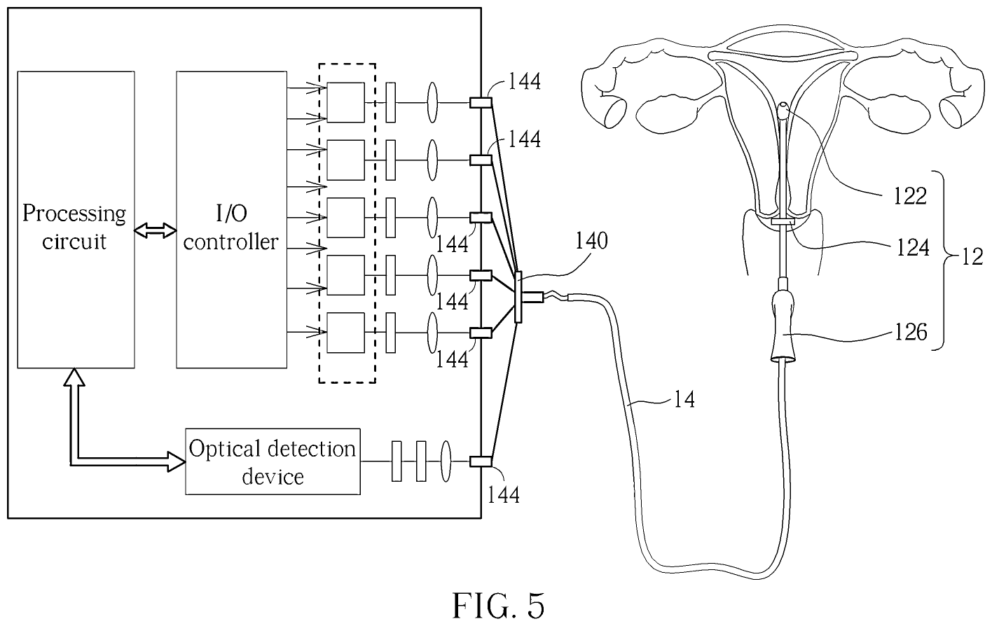

It should be noted that the detection device 10 is an embodiment of the present invention, and those with ordinary skill in the art may accordingly make different modifications, but not limited to this. Please refer to FIG. 5, which is a schematic diagram of the application of the detection device 10 to the optical detection system of US 2023/0059771 to realize the fiber module. The operation mode of the optical detection system of US 2023/0059771 is not described here for brevity. In this example, the optical fiber bundle 140 contains 6 optical fibers, and the connection terminal 144 also contains 6 optical fiber connectors. In addition, as shown in FIG. 5, because the optical fiber connectors are separable, the optical fiber bundle sheath 142 can fan out the optical fibers connected to the optical fiber connectors for easy connection to the optical detection system. Furthermore, when performing an examination of the endometrial mucosal tissue, the inspector may appropriately adjust the position of the positioning ring 124 to avoid the detection device 10 from going too deep and causing harm to the testee. At the same time, the smooth surface of the dome end 122 may also prevent harm to the testee, and the bend of the detection tube section 128 makes it easy to enter the uterine cavity through the cervical canal, which can also reduce the discomfort of the testee. Therefore, the detection device 10 can reduce the discomfort of the testee, reduce the difficulty of detection, and avoid causing harm to the testee. In this example, the target tissue area of the detection device 10 may be the endometrium located at the top of the uterus, the uterine cavity, the inner mouth of the cervix, the cervix or the outer mouth of the cervix, but not limited to this. The inspector may operate the detection device 10 to enter the cavity of the uterus so that the light generated by the light source device can be guided and irradiated to the endometrium through the detection device 10, and the reaction beams corresponding to the detection beams are also guided back to the light source device by the detection device 10. The wavelength range of the broadband light emitted by the light source device may be between 300 nanometers (nm) and 380 nm, 400 nm and 600 nm, 500 nm and 650 nm, 600 nm and 850 nm, 650 nm and 950 nm or 400 nm and 900 nm, and the detection device 10 can guide the broadband light emitted by the light source device almost without loss. The reaction beams generated by the reflection, Raman scattering, fluorescence or phosphorescence reaction after the specific biochemical molecules in the endometrium are excited by the light emitted by the light source device, the reaction beams are guided back to the optical sensing device by the optical fiber of the detection device 10 to make the optical sensing device detect the corresponding spectrum signals. Since the mucosal tissue contains many biomarker molecules with spontaneous fluorescence characteristics, such as NADP, NADPH, NADH, FAD, [CP(OME)3], [CP(ME)3], collagen, hemoglobin, porphyrin and porphyrin derivatives, the reaction beams are guided back to the optical sensing device by the detection device 10 to make the optical sensing g device analyze the reaction beams to generate the corresponding spectrum signals. The spectrum signals detected by the optical sensing device reflect the type and concentration of biological molecules in the mucosal tissue or mucosal tissue fluid. The processing circuit can determine the oxygen content, cell growth, mucosal inflammation, thickness and uniformity of the mucosal tissue based on the spectrum signals detected by the optical sensing device. In this way, the embodiment of the present invention can provide health parameters of real-time spectrum signals as a basis for judging health status and achieve real-time precise detection. Compared with the traditional endoscope method, the application of the optical detection system of the detection device 10 can greatly reduce the discomfort of the testee during the examination process. Furthermore, the traditional laser optical diagnosis method is to diagnose based on the image information of the mucosal surface. In contrast, the detection result of the embodiment of the present invention is not an unquantifiable image, but a specific quantifiable value, which can reduce the probability of errors caused by human eye judgment. The embodiments of the present invention may also provide real-time health parameters which are calculated and determined by utilizing spectrum signals and accurately utilized as reference values for early mucosal symptoms, thus significantly improving diagnostic accuracy.

Although FIG. 5 illustrates the detection of endometrium by the detection device 10 as an example, the mucosal tissue to be detected by the present invention is not limited thereto, and may also be the mucosa of the eye conjunctiva, mucosa, nasopharyngeal mucosa, respiratory mucosa, lung mucosa, esophageal mucosa, gastric wall mucosa, intestinal mucosa, urethral mucosa, reproductive mucosa, urinary tract, reproductive tract mucosa, etc., and is not limited thereto.

In summary, the detection device of the embodiment of the present invention has special structures, which can easily pass through the cervical canal into the uterine cavity, can reduce the discomfort of the testee, and reduce the difficulty of detection.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A detection device for an optical detection system, comprising:
an optical fiber catheter, having a feed opening and a dome end; and
an optical fiber detector, having one end coupled to the optical detection system through a connection terminal, and another end capable of entering an interior of the optical fiber catheter through the feed opening of the optical fiber catheter and reaching the dome end, configured to guide detection light beams generated by the optical detection system to the dome end, and guide reaction light beams corresponding to the detection light beams from the dome end to the optical detection system.

2. The detection device of claim 1, further comprising a positioning ring, set on the optical fiber catheter, wherein the positioning ring forms a detection tube section with the dome end, and forms an extension tube section with the feed opening, and the positioning ring is movable along the optical fiber catheter to adjust relative lengths of the detection tube section and the extension tube section.

3. The detection device of claim 2, wherein the detection tube section of the optical fiber catheter comprises a bend.

4. The detection device of claim 3, wherein an angle of the bend is selected from one of 0° to 15°, 15° to 30°, 30° to 45°, 45° to 60°, 60° to 75°, and 75° to 90°.

5. The detection device of claim 2, wherein the detection tube section of the optical fiber catheter is flexible and soft.

6. The detection device of claim 2, wherein the extension tube section of the optical fiber catheter is straight.

7. The detection device of claim 2, wherein an outer diameter of the positioning ring is smaller than or equal to 6 millimeters.

8. The detection device of claim 1, further comprising a handle, set on a position of the optical fiber catheter near the feed opening, wherein the handle does not obstruct the optical fiber detector from passing through the feed opening of the optical fiber catheter.

9. The detection device of claim 8, wherein the handle is gourd-shaped, and the handle includes a rough surface.

10. The detection device of claim 1, wherein the dome end is a hollow smooth spherical shape, and a diameter thereof is smaller than or equal to 3.0 millimeters.

11. The detection device of claim 1, wherein the dome end is a hollow smooth spherical shape, and a side of the dome end is a transparent film.

12. The detection device of claim 1, wherein a diameter of the optical fiber catheter is smaller than or equal to 3.0 millimeters.

13. The detection device of claim 1, wherein a total length of the optical fiber catheter is greater than 5 centimeters.

14. The detection device of claim 1, wherein at least one scale indicator is formed on the optical fiber catheter.

15. The detection device of claim 1, wherein the optical fiber catheter comprises a smooth surface.

16. The detection device of claim 1, wherein the optical fiber catheter is made of one or more of polytetrafluoroethylene, silicon, polyethylene, polypropylene, polymethylpentene, or polyvinyl chloride.

17. The detection device of claim 1, wherein the optical fiber detector comprises:
an optical fiber bundle, comprising a plurality of optical fibers, connected to the connection terminal; and
an optical fiber bundle sheath, which covers the optical fiber bundle;
wherein the connection terminal comprises a plurality of optical fiber connectors, corresponding to the plurality of optical fibers respectively.

18. The detection device of claim 17, wherein the plurality of optical fiber connectors are separated from each other, and the optical fiber bundle sheath allows the plurality of optical fibers connected to the plurality of optical fiber connectors to fan out and the plurality of optical fibers not connected to the plurality of optical fiber connectors in the optical fiber bundle to be closely bundled to pass through the feed opening of the optical fiber catheter.

19. The detection device of claim 17, wherein at least one of the plurality of optical fibers is used to guide the detection light beams generated by the optical detection system from at least one corresponding connection terminal to the dome end of the optical fiber catheter, and at least another one of the plurality of optical fibers is used to guide the reaction light beams corresponding to the detection light beams from the dome end to at least another corresponding connection terminal for output to the optical detection system.

20. The detection device of claim 17, wherein each of the plurality of optical fibers is a step-index multimode optical fiber, which comprises an optical fiber core, an optical fiber cladding, and a protective coating.
